(19) United States
(12) Patent Application Publication
(10) Pub. No.: US 2012/0035593 A1
(43) Pub. Date: Feb. 9, 2012

(54) ENTERAL CONNECTOR

(76) Inventor: Anthony C. LAIR, Alpharetta, GA (US)

(21) Appl. No.: 13/197,956

(22) Filed: Aug. 4, 2011

Related U.S. Application Data
(60) Provisional application No. 61/371,271, filed on Aug. 6, 2010.

(51) Int. Cl. A61M 25/16

(52) U.S. Cl. 604/535

(57) ABSTRACT

A male connector element for detachable coupling with an enteral feeding tube, and having a tip length sufficient to prevent over-insertion into the female hub of the feeding tube, and thereby reducing the potential for stretching the female hub.

### CROSS-REFERENCE TO RELATED APPLICATION

This application claims the benefit of U.S. Provisional Patent Application Ser. No. 61/371,271, filed Aug. 6, 2010, the entirety of which is hereby incorporated herein by reference for all purposes.

### TECHNICAL FIELD

The present invention relates generally to the field of medical devices and procedures, and more particularly to an improved connector for enteral, nasogastric and/or gastric feeding tubes, extension tubes, and/or other fluid delivery conduit connections, as well as to associated systems and methods utilizing such a connector.

### BACKGROUND

Enteral feeding tubes are used to provide nutrition to human or animal patients who can not obtain sufficient nutrition by eating through the mouth by swallowing. Nasogastric feeding tubes are inserted through the nares (nostril), through the esophagus, and into the stomach of the patient. Gastric feeding tubes may be percutaneously inserted into the stomach. As used herein, enteral, nasogastric, gastric and other nutrient delivery components are collectively referred to as enteral components. The distal or discharge end of the feeding tube is positioned internally and delivers liquid nutrients into the stomach, while the proximal end of the feeding tube is positioned externally for connection to a nutrient source.

Once placed, a feeding tube may remain in the patient for several weeks or more, depending upon the continued need for enteral feeding; whereas the nutrient source is typically replaced more frequently, for example on a daily basis. For example, in enteral feeding of infants, the nutrient source may be a container of breast milk or formula, which is replaced one or more times per day. To facilitate replacement of the nutrient source without disturbing the placement of the feeding tube, the proximal or external end of a feeding tube commonly comprises a female enteral connector hub into which a male connector element or enteral tip may be removably coupled, typically by an interference fit resulting from a progressive taper provided on the enteral tip.

Repeated replacement of the nutrient source can lead to stretching of the female enteral connector hub by the enteral tip, potentially resulting in loss of the interference fit required for a reliable connection at the enteral coupling between the hub and tip. Over-insertion of the male enteral tip into the female connector hub can exacerbate the potential for stretching and permanent deformation of the hub, due to the taper of the enteral tip having an increasing diameter the further it is inserted. Loss of a reliable connection at the enteral coupling typically necessitates replacement of the feeding tube, which may be traumatic for some patients or interfere with a normal feeding regimen, and is generally undesirable.

Accordingly, it can be seen that needs exist for improvements to enteral feeding systems and methods. It is to the provision of an improved enteral connector and to improved feeding systems and methods meeting these and other needs that the present invention is primarily directed.

### SUMMARY

In example embodiments, the present invention provides an improved connector for enteral feeding. In one aspect, the present invention relates to a male connector element for coupling with a female connector hub. The male connector element includes a tip portion having a tip length substantially greater than a receiver length of the female connector hub, thereby preventing over-insertion.

In another aspect, the invention relates to an enteral connector or extension tube. The enteral connector tube includes a male connector element affixed to a first end of a tube and a female connector element affixed to a second end of the tube. The male connector element includes a tip portion having a tip length substantially greater than a corresponding receiver length of a female connector hub, thereby preventing over-insertion into the female hub.

In another aspect, the invention relates to an enteral coupling comprising a male connector element releasably coupled to a cooperating female connector hub. The male connector element includes a tip portion having a tip length substantially greater than a corresponding receiver length of a female connector hub, thereby preventing over-insertion into the female hub.

In still another aspect, the invention relates to a male connector element having a coupling segment for detachable coupling with a female receiver. The coupling segment defines a male coupling length in an axial direction and a male coupling outer dimension transverse to the axial dimension. The female receiver defines a female coupling length in the axial direction and a female coupling outer dimension transverse to the axial dimension. The male coupling length is greater than the female coupling length, and the male coupling outer dimension and the female coupling outer dimension are selected to form a sealing engagement therebetween without exceeding an elastic deformation limit of the female receiver.

In another aspect, the invention relates to a method of detachably coupling a male connector with a female connector. The method includes insertion of a coupling segment of the male connector into a receiver of the female connector to an insertion depth. The receiver of the female connector defines a female coupling length in an axial dimension of no greater than the insertion depth, and the coupling segment of the male connector defines a male coupling length in the axial dimension of greater than the insertion depth.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of an enteral connector and delivery tube according to an example embodiment of the present invention.

FIG. 2 shows the enteral connector of FIG. 1 and a female connector hub, in an unconnected or detached state.

FIG. 3 shows the enteral connector of FIG. 1 partially inserted into the female connector hub.

FIG. 4 shows the enteral connector of FIG. 1 partially inserted into the female connector hub, with the female connector hub partially cut away to show the internal connection.

FIG. 5 shows the enteral connector of FIG. 1 further inserted into the female connector hub.

FIG. 6 shows the enteral connector of FIG. 1 fully inserted into and engaged with the female connector hub.

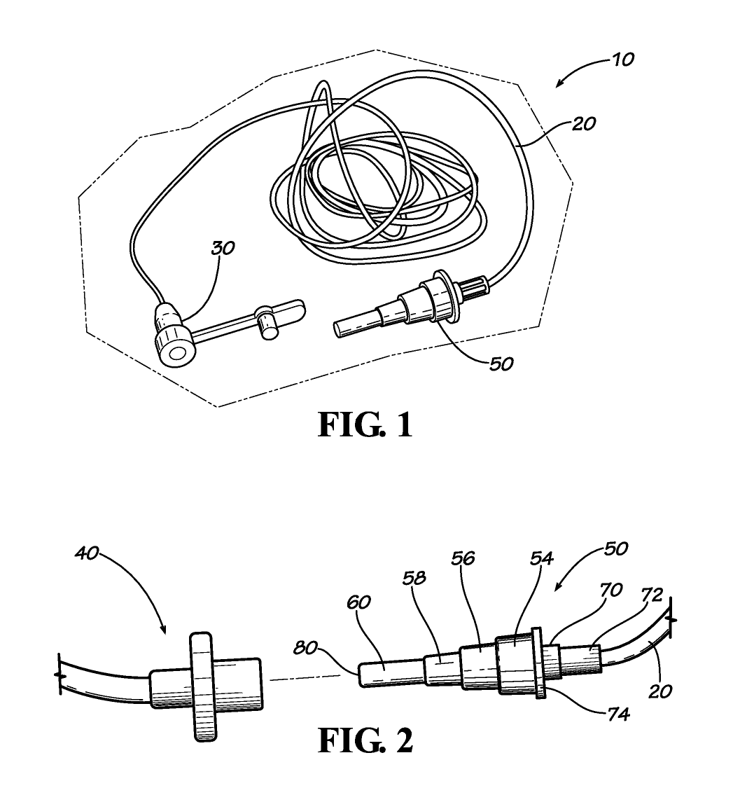

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

With reference now to the drawing figures, wherein like reference numbers represent corresponding parts throughout the several views, an enteral connector tube 10 according to an example embodiment of the invention generally comprises a flexible tube or delivery conduit 20 having a female connector element or coupling member 30 at one end and a male connector element or coupling member 50 at the other end. The female connector element 30 is configured for removable attachment to, for example, a metered syringe, dispenser, bag, or other container of breast milk, formula or other nutrient source.

The male connector element or coupling member 50 comprises a stepped profile having four segments 54, 56, 58, 60 of progressively decreasing diameter moving from its proximal end to distal tip. In alternate embodiments, one or both of the intermediate steps or segments 56, 58 may optionally be omitted, resulting in a two or three-segmented coupling member. The proximal end of the male coupling member 50 comprises a receiver 70 for attachment of the tube 20, optionally including a strain-relief bushing 72, and a flange 74. An internal bore or channel extends axially and lengthwise through the male coupling member 50, providing fluid communication from the tube 20 through a discharge opening 80 at the tip of the distal segment 60.

The distal tip portion or coupling segment 60 of the male coupling member 50 preferably tapers slightly (about 1°-3° of taper, preferably about 1.25°) from a larger outside diameter at the intersection with third segment 58 to a smaller outside diameter of about 4.5 mm-5.0 mm, preferably about 4.7 mm, at its tip, and has a radiused outer chamfer at the tip. The distal segment 60 preferably has a length L_{M} of at least about 10 mm, and preferably at least about 12 mm (for example about 12.5 mm in the depicted embodiment), or in any event a length L_{M} greater than the length L_{F} of the female receiver into which the male coupling member 50 is to be connected (see FIG. 4). For example, if a female length L_{F} of about 8.5 mm is adopted as a standard for a particular coupling set, the male length L_{M} would be at least about 8.5-9.0 mm or more.

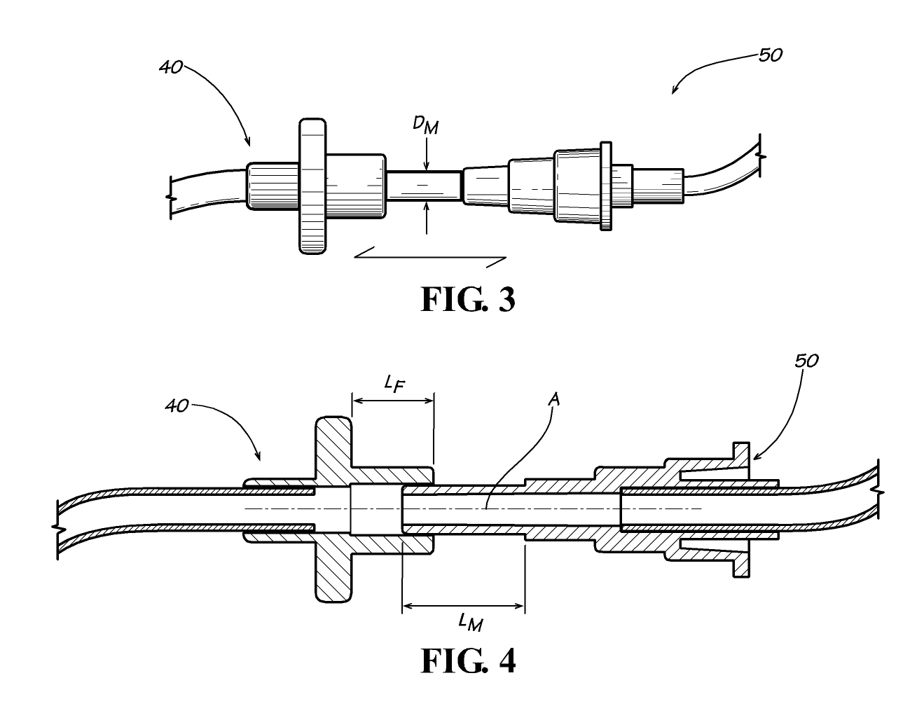

FIGS. 3-6 show a sequence of progressive axial (along axis A as shown in FIG. 4) insertion of the male coupling member 50 into the receiver of a cooperating female coupling hub 40. The depicted form of female hub 40 is by way of example only, and the male coupling member of the invention is preferably adaptable for universal use with different types or formats of female hub components produced by various manufacturers. The female hub 40 is, for example, the proximal end of an enteral feeding tube positioned external of a patient, and having its distal or discharge end positioned internally to deliver liquid nutrients into the patient's stomach.

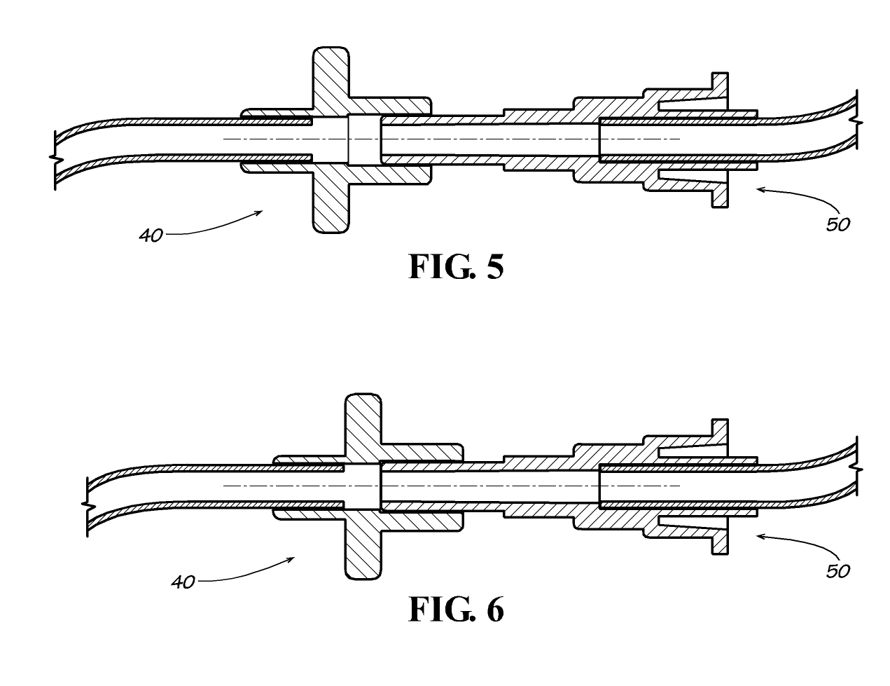

The male coupling member 50 may be inserted into the receiver of the female hub 40 as shown in the sequence of FIG. 4 (partially inserted) to FIG. 5 (further inserted) to FIG. 6 (fully inserted). Typically it will be unnecessary to fully insert the male coupling member, as an adequate interference fit connection will be obtained before full insertion. The male coupling member is disconnected and removed from the female hub by reversing the sequence of operation.

Progressive insertion of the male coupling member 50 into the receiver of the cooperating female hub or coupling member 40 requires progressively increasing application of force, due to the tapered profile of the distal or tip segment 60 of the male coupling member. Over-insertion of the male coupling member into the receiver of the cooperating female hub or coupling member is prevented by means of the length L_{M} of the distal segment 60 of the male coupling member being greater than the length L_{F} of the receiver chamber of the female coupling member. As such, the tip of the male coupling member abuts a shoulder or other stop member within the receiver chamber of the female coupling member before the larger diameter third segment 58 of the male coupling member enters the receiver of the female coupling member.

The outer diameter D_{M} or (other outer dimension transverse to the axis A, if the coupling member is not circular) and degree of taper of the distal segment 60 of the male coupling member are selected to provide an interference fit and sealing connection within the inner diameter of the receiver chamber of the female coupling member without exceeding the elastic deformation limit of the female coupling member, when inserted to its maximum depth, so as not to produce a permanent deformation of the female coupling member which could result in loss of a reliable connection leading to the need for replacement of a feeding tube.

While the invention has been described with reference to preferred and example embodiments, it will be understood by those skilled in the art that a variety of modifications, additions and deletions are within the scope of the invention, as defined by the following claims.

What is claimed is:

1. A male connector element for coupling with a female connector hub, the male connector element comprising a tip portion having a tip length substantially greater than a receiver length of the female connector hub.

2. The male connector element of claim 1, wherein the male connector element and the female connector hub comprise an enteral coupling.

3. The male connector element of claim 1, wherein the tip length is at least about 10 mm.

4. The male connector element of claim 1, wherein the tip length is at least about 12 mm.

5. The male connector element of claim 1, wherein the tip length is about 12.5 mm.

6. The male connector element of claim 1, wherein the tip portion has a tapered outer diameter forming an interference fit with the receiver of the female connector hub.

7. The male connector element of claim 1, having a stepped outer profile including at least two segments of progressively decreasing dimension toward the tip portion.

8. The male connector element of claim 1, wherein the stepped outer profile comprises four segments.

9. An enteral connector tube comprising the male connector element of claim 1 affixed to a first end of a tube, and a female connector element affixed to a second end of the tube.

10. An enteral coupling comprising the male connector element of claim 1 releasably coupled to a cooperating female connector hub.

11. A male connector element having a coupling segment for detachable coupling with a female receiver, the coupling segment defining a male coupling length in an axial direction and a male coupling outer dimension transverse to the axial dimension, the female receiver defining a female coupling length in the axial direction and a female coupling outer dimension transverse to the axial dimension, wherein the male coupling length is greater than the female coupling length, and wherein the male coupling outer dimension and the female coupling outer dimension are selected to form a sealing engagement therebetween without exceeding an elastic deformation limit of the female receiver.

12. The male connector element of claim 11, wherein the male coupling length is at least about 10 mm.

13. The male connector element of claim 11, wherein the male coupling length is at least about 12 mm.

14. The male connector element of claim 11, wherein the male coupling length is about 12.5 mm.

15. The male connector element of claim 11, wherein the male coupling segment has a tapered outer diameter that decreases toward a distal end thereof.

16. The male connector element of claim 11, having a stepped outer profile including at least two segments of progressively decreasing dimension toward a distal end thereof.

17. The male connector element of claim 16, wherein the stepped outer profile comprises four segments.

18. An enteral connector tube comprising the male connector element of claim 11 affixed to a first end of a tube, and a female connector element affixed to a second end of the tube.

19. An enteral coupling comprising the male connector element of claim 11 releasably coupled to a cooperating female connector hub comprising the female receiver.

20. A method of detachably coupling a male connector with a female connector, the method comprising insertion of a coupling segment of the male connector into a receiver of the female connector to an insertion depth, wherein the receiver of the female connector defines a female coupling length in an axial dimension of no greater than the insertion depth, and wherein the coupling segment of the male connector defines a male coupling length in the axial dimension of greater than the insertion depth.

21. The method of claim 20, wherein insertion of the coupling segment of the male connector into the receiver of the female connector forms a sealing engagement therebetween without exceeding an elastic deformation limit of the receiver.

22. The method of claim 20, further comprising delivery of a fluid through the coupling formed by the male connector and the female connector.
